(19) United States
(12) Patent Application Publication
(10) Pub. No.: US 2019/0310293 A1
(43) Pub. Date: Oct. 10, 2019

(54) Method for Detecting Normal Harmonics of Earths Electromagnetic Atmospheric Resonant Frequency First Harmonic of 15.3 Hz and Abnormal Frequencies Corresponding to Neurological Diseases

(71) Applicant: Andres Herbert Montoya Urquizo, Lima (PE)

(72) Inventor: Andres Herbert Montoya Urquizo, Lima (PE)

(21) Appl. No.: 16/380,906

(22) Filed: Apr. 10, 2019

(30) Foreign Application Priority Data
Apr. 10, 2018 (PE) 000535-2018/DIN

(51) Int. Cl. G01R 23/20; G01R 23/177; A61B 5/04

(52) U.S. Cl. CPC G01R 23/20; A61B 5/04005; G01R 23/177

(57) ABSTRACT

A detection method is provided to detect the normal harmonics of the first harmonic of Earth's atmospheric electromagnetic resonance frequency of 15.3 Hz and abnormal frequencies of corresponding to neurological diseases. The method will assist and guide neurological specialists in providing a good treatment to the patients in order to reduce the abnormal frequencies in such a way as to control the disease and maintain a balanced well-being of the patient.

### BACKGROUND OF THE INVENTION

In 1952 German physicist Winfried Schumann discovered the Earth's atmospheric electromagnetic resonance frequency, a wave with a frequency of 7.83 Hz that oscillates between the surface of the Earth and the ionosphere between 90 and 500 km from the surface of the Earth, as illustrated in FIG. 1a. The distance/space between Earth and its ionosphere acts as a resonant cavity for the electromagnetic waves in the ELF band (extremely low frequency).

It has been proven that this wave resonates with man and all mammalian animals through the hypothalamus of the brain, an organ that retransmits this frequency and its harmonics (i.e., multiples of that frequency), creating a healthy biological relationship between man and Earth.

Earth's atmospheric electromagnetic resonance frequency appears accompanied by its harmonics as illustrated in FIGS. 1b and 1c (i.e., multiple of that frequency), frequencies that have been detected by world scientists, being the first harmonic at 14.1 Hz the one with the highest intensity. These Figures also illustrates the way some frequencies bounce between the ionosphere and the Earth surface according to its wavelength.

### BRIEF DESCRIPTION OF THE DRAWINGS

Further features and advantages of the invention will become apparent from the following detailed description taken in conjunction with the accompanying figures showing illustrative embodiments of the invention, in which:

FIG. 1a illustrates how Earth's resonant frequency is reflected between the ionosphere and the ground.

FIG. 1b illustrates different harmonics of Earth's resonant frequency around Earth.

FIG. 1c is a plot representing Earth's resonant frequency along with its different harmonics.

FIG. 2 illustrates the system used to perform the detection method according to the present invention.

FIG. 3 is a plot illustrating the resonant frequency in relation to its harmonics and abnormal frequencies according to the present invention.

FIG. 4 is a graphical illustration of the 15.3 Hz frequency as detected and shown in an oscilloscope.

FIG. 5 is a table showing several diseases correlating to certain detected frequencies.

FIG. 6 is a table showing common abnormal frequencies among several diseases.

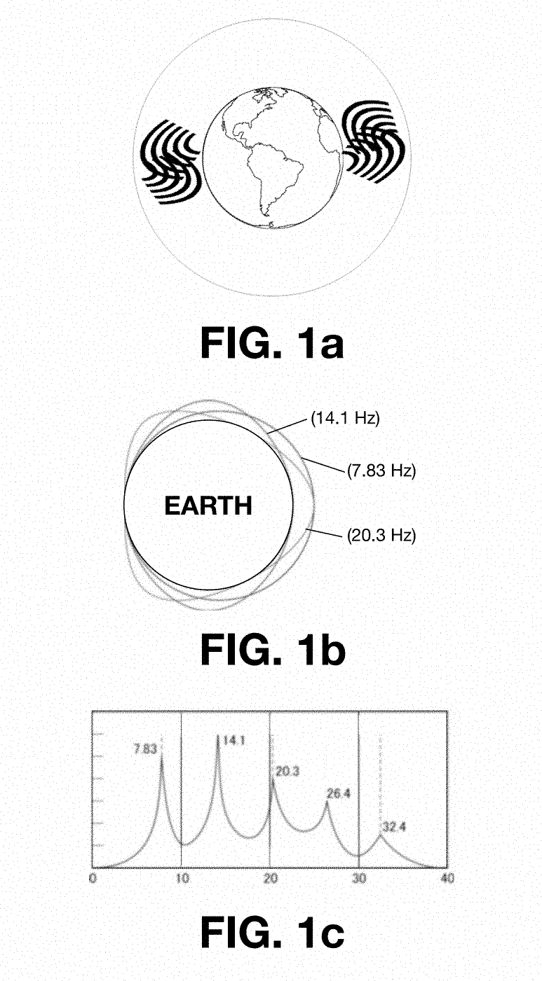

### DETAILED DESCRIPTION OF THE INVENTION

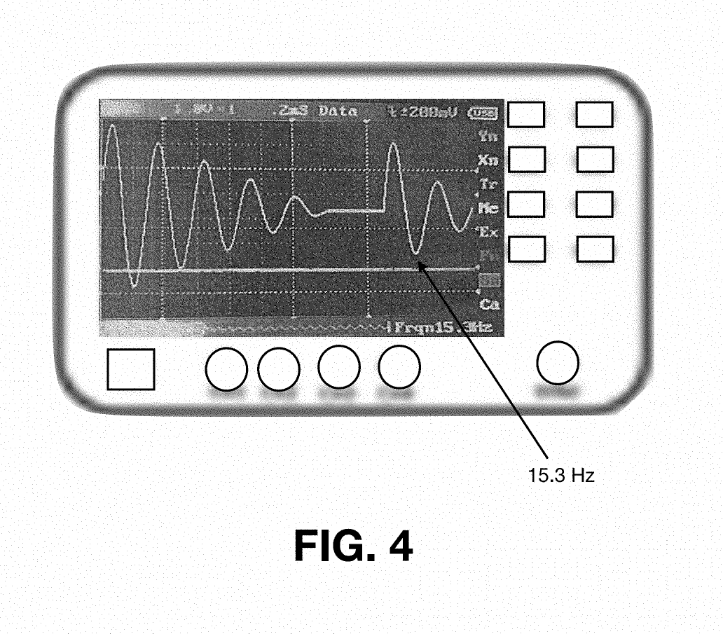

The method of the present invention allowed me to initially detect the appearance of a 15.3 Hz frequency (FIG. 4) with the assistance of a mini-oscilloscope 3 (instrument used to observe and measure current, voltage and frequency of a wave) and a protoboard 4 (plate that allows to install and interconnect electrical or electronic circuits without welding), having only one cable installed and without electrical power connected to the tip of the oscilloscope 3. This 15.3 Hz frequency turned out to be the first harmonic of the Earth's atmospheric electromagnetic resonance frequency, which was the same frequency detected in Lima (Peru), Barcelona (Spain) and Jerusalem (Israel). This harmonic frequency is the most intense in the world with a value that has varied with reference to the frequency detected (14.1 Hz) many years ago.

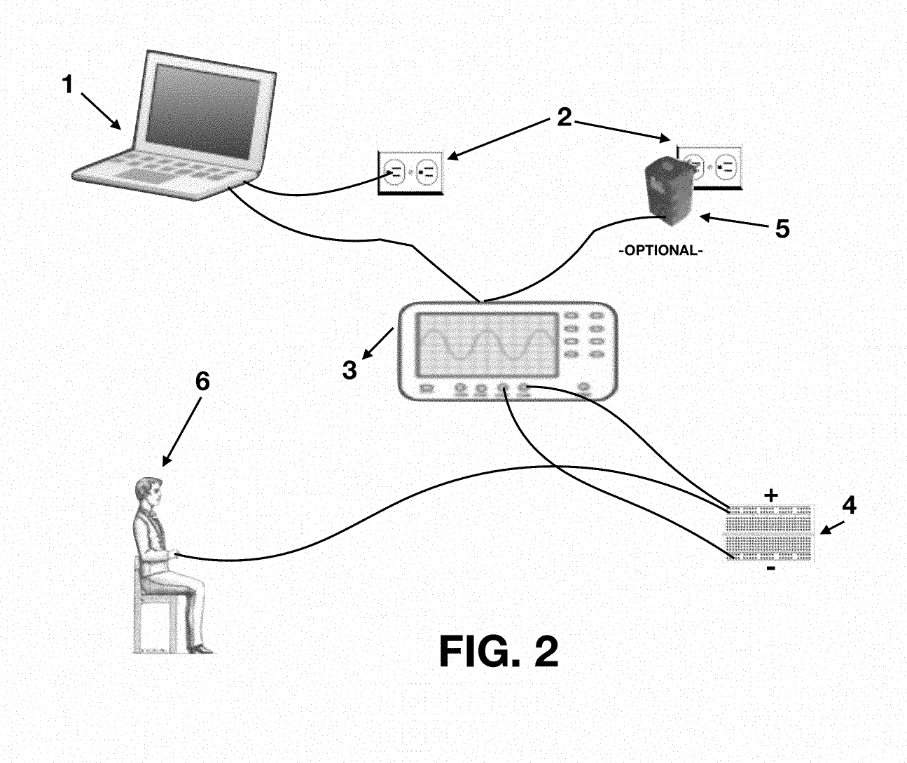

In order to carry out the experiment according to the present invention as shown in FIG. 2, it was necessary to connect the small oscilloscope 3 (through the USB input) to a power source. This was done by either using an AC/DC electrical adapter 5 outputting 5 V_{dc}, or a USB output of the laptop or personal computer 1 connected to a 220/110 V_{ac} power outlet 2 at approximately 60/50 Hertz so that an electric field generated triggers the appearance and posterior detection of the 15.3 Hz frequency. When a cable was touched at its end by a person 6, it was observed that the frequencies 76.3 Hz and 61 Hz appeared initially intermittently at the heart beat rate, being the constant frequency of 15.3 Hz the difference between the two frequencies. It must be noted that the value at the peak of the pulse rise, corresponds to the 15.3 Hz frequency (first harmonic of the Earth's atmospheric electromagnetic resonance frequency) that passes in synchronization through the hypothalamus of our brain plus 61 Hz, which turned out to be a detection constant generated by the electric field when the laptop or PC 1 is connected to the commercial electrical network 2. However, at the peak of the cardiac pulse fall, the aforementioned harmonic frequency is not registered by our body, only the 61 Hz frequency appears which is the initial detection constant.

Given that the human body's electrical resistance characteristics depend from person to person, the electrical resistance can vary from 0 to 3000 ohms throughout the day. In the case of the inventor, greater resistive effect was observed during a certain time of the day which allowed not only to register the above-mentioned harmonic frequencies, but also the appearance of the following frequencies that are multiples of the first harmonic (15.3 Hz) of the earth's atmospheric electromagnetic resonance frequency and thus, also harmonics of the latter frequency according to the following formula:

$$Y = \frac{( X - 61 )}{15.3} , \qquad ( Eq . 1 )$$

where X is the detected frequency in Hz, Y is the harmonic corresponding to the detected frequency (multiple or submultiple of 15.3 Hz).

This equation remains valid throughout the planet, since the first harmonic of 15.3 Hz remained constant in several parts of the world (Peru, Spain and Israel) regardless of the commercial electric frequency of 50 or 60 Hz from the country. This confirms that the ionospheric variation has no influence on the recorded detection of harmonic frequencies in other latitudes of the world.

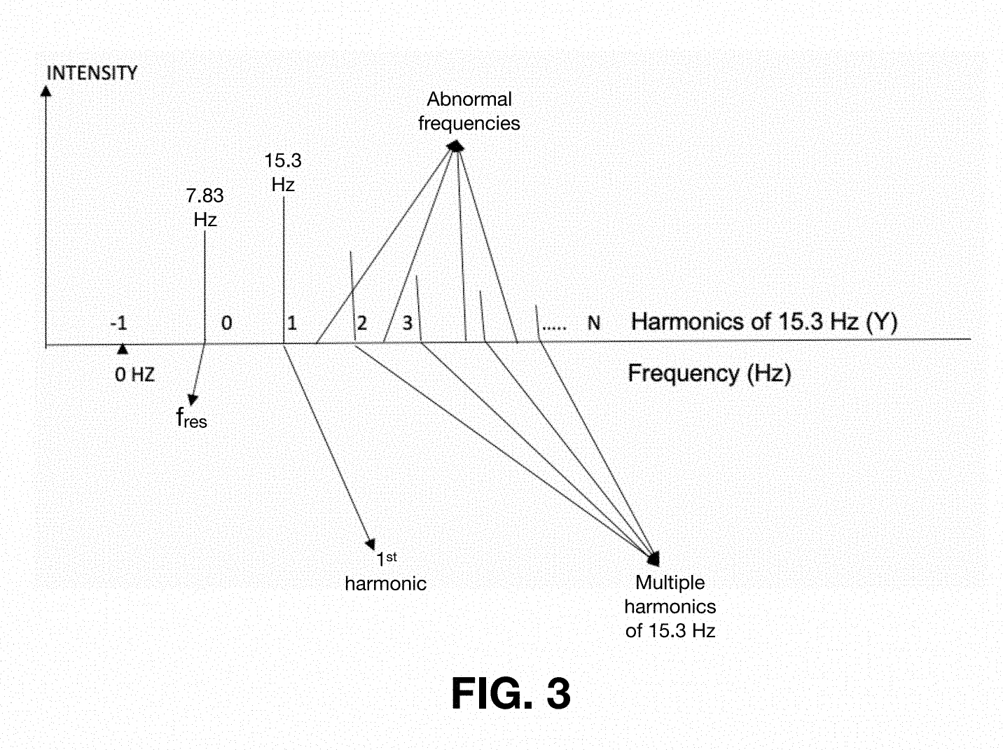

To calculate the multiple of 15.3 Hz, the detected frequency X is subtracted from the 61 Hz, and divided by 15.3 Hz, rounded up to the next integer (from x.90 to x.99) for the case of healthy people. If the number is lower than this value range, then it is considered “as is” with decimals, which will identify an abnormality or a nervous alteration (people with neurological diseases), see FIG. 3.

The experiments carried out following the inventive procedure were voluntarily performed on adults and children in healthy condition and with neurological diseases to support the tests of this experiment with the following obtained results.

### Healthy Adults:

1. Inventor, male, 61 years old, November 2017, 10 a.m., Lima, Perú. Twenty (20) normal harmonics were detected: −1, 0, 1, 2, 3, 4, 5, 6, 7, 8, 9, 10, 11, 12, 13, 14, 15, 16, 17, 19.

| X - Detected | |
|---|---|
| Frequency | |
| (0-200 Hz) | Y - Harmonic |
| 45.8 | −0.99 = −1 |
| 61.0 | 0 |
| 76.3 | 1 |
| 91.6 | 2 |
| 107 | 3 |
| 122 | 3.98 = 4 |
| 137 | 4.96 = 5 |
| 153 | 6.01 = 6 |
| 198 | 8.95 = 9 |
| 168 | 6.99 = 7 |
| 183 | 7.97 = 8 |

| X - Detected | |
|---|---|
| Frequency | |
| (200-400 Hz) | Y - Harmonic |
| 214 | 10 |
| 229 | 10.98 = 11 |
| 244 | 11.96 = 12 |
| 259 | 12.94 = 13 |
| 275 | 13.98 = 14 |
| 290 | 14.96 = 15 |
| 305 | 15.94 = 16 |
| 320 | 16.92 = 17 |
| 351 | 18.95 = 19 |

2. Inventor, male, 61 years old, November 2017, 5 p.m., Lima, Perú. Fourteen (14) normal harmonics were detected: 11, 12, 17, 19, 20, 21, 22, 23, 24, 27, 28, 29, 30, 31.

| X - Detected | |
|---|---|
| Frequency | |
| (0-300 Hz) | Y - Harmonic |
| 244 | 11.96 = 12 |
| 229 | 10.98 = 11 |

| X - Detected | |
|---|---|
| Frequency | |
| (300-600 Hz) | Y - Harmonic |
| 443 | 24.96 = 25 |
| 351 | 18.95 = 19 |
| 320 | 16.92 = 17 |
| 412 | 22.94 = 23 |
| 366 | 19.93 = 20 |
| 397 | 21.96 = 22 |
| 534 | 30.91 = 31 |
| 504 | 28.95 = 29 |
| 519 | 29.93 = 30 |
| 427 | 23.92 = 24 |
| 473 | 26.92 = 27 |
| 381 | 20.91 = 21 |
| 488 | 27.90 = 28 |

3. Male subject, 63 years old, Nov. 21, 2017, 5 p.m., Lima, Perú. Fourteen (14) normal harmonics were detected: −1, 0, 1, 2, 3, 4, 6, 7, 9, 10, 11, 12, 13, 14.

| X - Detected | |
|---|---|
| Frequency | |
| (300-600 Hz) | Y - Harmonic |
| 61 | 0 |
| 76.3 | 1 |
| 45.8 | −0.99 = −1 |
| 107 | 3 |
| 122 | 3.98 = 4 |
| 168 | 6.99 = 7 |
| 91.6 | 2 |
| 153 | 6.01 = 6 |
| 198 | 8.95 = 9 |
| 259 | 12.94 = 13 |
| 214 | 10 |
| 275 | 13.98 = 14 |
| 229 | 10.98 = 11 |
| 244 | 11.96 = 12 |

4. Female subject, 39 years old, Nov. 21, 2017, 6 p.m., Lima, Perú. Ten (10) normal harmonics were detected: 1, 2, 3, 4, 5, 6, 7, 9, 11, 13.

| X - Detected | |
|---|---|
| Frequency (Hz) | Y - Harmonic |
| 137 | 4.96 = 5 |
| 168 | 6.99 = 7 |
| 76.3 | 1 |
| 107 | 3 |
| 122 | 3.98 = 4 |
| 91.6 | 2 |
| 153 | 6.01 = 6 |
| 198 | 8.95 = 9 |
| 259 | 12.94 = 13 |
| 229 | 10.98 = 11 |

5. Female subject, 97 years old, Nov. 1, 2017, 3 p.m., Lima, Perú. Three (3) normal harmonics were detected: −1, 0, 1.

| X - Detected | |
|---|---|
| Frequency (Hz) | Y - Harmonic |
| 61 | 0 |
| 76.3 | 1 |
| 45.8 | −0.99 = −1 |

6. Female subject, 61 years old, Jan. 5, 2018, 10 a.m., Lima, Perú. Nine (9) normal harmonics were detected using the right hand: 0, 1, 2, 3, 4, 5, 6, 7, 13. Fourteen (14) normal harmonics were detected using the left hand: −1, 1, 2, 3, 4, 5, 6, 8, 9, 10, 13, 14, 15, 20.

### Right Hand

| X - Detected | |
|---|---|
| Frequency (Hz) | Y - Harmonic |
| 91.6 | 2 |
| 76.3 | 1 |
| 137 | 4.96 = 5 |
| 61 | 0 |
| 259 | 12.94 = 13 |
| 107 | 3 |
| 153 | 6.01 = 6 |
| 168 | 6.99 = 7 |
| 122 | 3.98 = 4 |

### Left Hand

| X - Detected | |
|---|---|
| Frequency (Hz) | Y - Harmonic |
| 107 | 3 |
| 91.6 | 2 |
| 122 | 3.98 = 4 |
| 76.3 | 1 |
| 45.8 | −0.99 = −1 |
| 198 | 8.95 = 9 |
| 137 | 4.96 = 5 |
| 214 | 10 |
| 259 | 12.94 = 13 |
| 153 | 6.01 = 6 |
| 366 | 19.93 = 20 |
| 275 | 13.98 = 14 |
| 290 | 14.96 = 15 |
| 183 | 7.97 = 8 |

7. Inventor, 61 years old, Jan. 15, 2018, 10 a.m., Lima, Perú. Twenty-one (21) normal harmonics were detected using the right hand: 1, 2, 3, 4, 5, 6, 7, 8, 9, 10, 11, 13, 14, 15, 17, 18, 19, 22, 23, 28, 29. Twenty eight (28) normal harmonics were detected using the left hand: 3, 4, 5, 6, 7, 8, 9, 10, 11, 12, 13, 14, 15, 16, 17, 18, 19, 20, 21, 22, 23, 24, 25, 26, 31, 32, 33, 34.

### Right Hand

| X - Detected | |
|---|---|
| Frequency (Hz) | Y - Harmonic |
| 183 | 7.97 = 8 |
| 91.6 | 2 |
| 168 | 6.99 = 7 |
| 107 | 3 |
| 153 | 6.01 = 6 |
| 259 | 12.94 = 13 |
| 198 | 8.95 = 9 |
| 122 | 3.98 = 4 |
| 229 | 10.98 = 11 |
| 488 | 27.90 = 28 |
| 290 | 14.96 = 15 |
| 336 | 17.97 = 18 |
| 504 | 28.95 = 29 |
| 351 | 18.95 = 19 |
| 397 | 21.96 = 22 |
| 214 | 10 |
| 412 | 22.94 = 23 |
| 275 | 13.98 = 14 |
| 320 | 16.92 = 17 |
| 137 | 4.96 = 5 |
| 76.3 | 1 |

### Left Hand

| X - Detected | |
|---|---|
| Frequency (Hz) | Y - Harmonic |
| 153 | 6.01 = 6 |
| 198 | 8.95 = 9 |
| 168 | 6.99 = 7 |
| 137 | 4.96 = 5 |
| 229 | 10.98 = 11 |
| 183 | 7.97 = 8 |
| 214 | 10 |
| 107 | 3 |
| 122 | 3.98 = 4 |
| 336 | 17.97 = 18 |
| 259 | 12.94 = 13 |
| 275 | 13.98 = 14 |
| 580 | 33.92 = 34 |
| 305 | 15.94 = 16 |
| 366 | 19.93 = 20 |
| 244 | 11.96 = 12 |
| 412 | 22.94 = 23 |
| 290 | 14.96 = 15 |
| 351 | 18.95 = 19 |
| 336 | 17.97 = 18 |
| 443 | 24.96 = 25 |
| 320 | 16.92 = 17 |
| 427 | 23.92 = 24 |
| 534 | 30.91 = 31 |
| 549 | 31.89 = 32 |
| 397 | 21.96 = 22 |
| 381 | 20.91 = 21 |
| 565 | 32.94 = 33 |
| 458 | 25.94 = 26 |

8. Female subject, left-hander, 31 years old, Feb. 7, 2018, 7 p.m., Lima, Perú. Three (3) normal harmonics were detected using the right hand: −1, 0, 1. Three (3) normal harmonics were detected using the left hand: −1, 0, 1.

### Right Hand

| X - Detected | |
|---|---|
| Frequency (Hz) | Y - Harmonic |
| 61 | 0 |
| 76.3 | 1 |
| 45.8 | −0.99 = −1 |

### Left Hand

| X - Detected | |
|---|---|
| Frequency (Hz) | Y - Harmonic |
| 61 | 0 |
| 76.3 | 1 |
| 45.8 | −0.99 = −1 |

9. Male subject, 60 years old, Feb. 22, 2018, 3 p.m., Barcelona, Spain. Twenty-one (21) normal harmonics were detected using the right hand: −1, 0, 1, 2, 3, 4, 5, 11, 13, 15, 16, 17, 19, 21, 22, 26, 27, 31, 33, 35, 45.

### Right Hand

| X - Detected | |
|---|---|
| Frequency (Hz) | Y - Harmonic |
| 76.3 | 1 |
| 229 | 10.98 = 11 |
| 45.8 | −0.99 = −1 |
| 565 | 32.94 = 33 |
| 137 | 4.96 = 5 |
| 473 | 26.92 = 27 |
| 351 | 18.95 = 19 |
| 534 | 30.91 = 31 |
| 320 | 16.92 = 17 |
| 381 | 20.91 = 21 |
| 259 | 12.94 = 13 |
| 91.6 | 2 |
| 107 | 3 |
| 61.0 | 0 |
| 305 | 15.94 = 16 |
| 595 | 34.90 = 35 |
| 458 | 25.94 = 26 |
| 397 | 21.96 = 22 |
| 565 | 32.94 = 33 |
| 748 | 44.90 = 45 |
| 290 | 14.96 = 15 |
| 122 | 3.98 = 4 |

10. Female subject, 57 years old, Mar. 3, 2018, 3 p.m., Jerusalem, Israel. Six (6) normal harmonics were detected using the right hand: 1, 3, 4, 5, 6, 7.

### Right Hand

| X - Detected | |
|---|---|
| Frequency (Hz) | Y - Harmonic |
| 107 | 3 |
| 137 | 4.96 = 5 |
| 122 | 3.98 = 4 |
| 76.3 | 1 |
| 168 | 6.99 = 7 |
| 153 | 6.01 = 6 |

As can be appreciated in the Case 1, I am 61 years old and the experiment was carried out on Nov. 17, 2017 in the morning. All the harmonics Y are multiples of 15.3 Hz (the first harmonic of 7.83 Hz, Earth's atmospheric electromagnetic resonance frequency) except the harmonic −1 which is a submultiple of that frequency (corresponding to 0 Hz). The group of recorded harmonic frequencies that are multiples of 15.3 Hz was: −1, 0, 1, 2, 3, 4, 5, 6, 7, 8, 9, 10, 11, 12, 13, 14, 15, 16, 17, 19.

I repeated this experiment on Nov. 21, 2017 at 5:00 p.m. obtaining additional frequencies. This time, apart from harmonics and 12 obtained previously, major frequency harmonics were registered: 17, 19, 20, 21, 22, 23, 24, 25, 27, 28, 29, 30, 31.

This set of frequencies, of both experiments, turned out to be common to the frequencies detected in adults with a healthy condition (whose numbers are integers by approximation and multiples of 15.3 Hz), as previously shown.

In the Case 5 for the female subject (97 years old) in good health condition, it can be appreciated that in the experiment conducted on Nov. 1, 2017 at 3:00 p.m. the obtained frequencies 76.3, 45.8 and 61 Hz correspond to the harmonics 1, −1 and 0. These are only 3 frequencies for an elderly female who is at permanent rest, results that are very similar to those obtained for the female children subjects of 3 and 6 years old in Cases 1 and 2, respectively, as will be explained below.

In Case 6, the female subject was tested using the right hand and the left hand, where more normal harmonics were obtained with the left hand. It can be inferred that by being right-handed, the left lobe of the brain is the predominant side for reasoning and control purposes, dealing with speech, writing, numeration, mathematics and logic with a smaller amount of normal harmonic frequencies. In contrast, the right hemisphere is in charge of feelings, emotions, creativity, art and music skills, with a greater presence of harmonics that indicate the potential to develop these qualities and skills in the person. The results of the experiment of Case 7 performed on myself, supports the same inferences as in Case number 6 previously explained.

In Case 8 for the female subject (left-handed), the test showed the same number of normal frequencies harmonics in the right hand and the left hand, contrasting with the other cases where a greater number of harmonics was obtained in the left hand.

In Case 9, the test was carried out using the right hand of the male subject in the city of Barcelona, Spain, verifying that the value of 15.3 Hz (first harmonic of the Earth's resonance frequency) remained constant in this place. Likewise, the harmonic multiples of 15.3 Hz appeared as normal frequencies given the subject's healthy condition.

In case 10, the female subject did the test with the right hand in the city of Jerusalem, Israel, also verifying that the value of 15.3 Hz (first harmonic of the Earth's resonance frequency) remained constant in this place. Likewise, the harmonic multiples of 15.3 Hz appeared as normal frequencies given the subject's healthy condition.

### Healthy Children (Under Parents' Consent):

1. Female subject, 3 years old, Nov. 1, 2017, 10 a.m., Lima, Perú. Six (6) normal harmonics were detected using the right hand: −1, 0, 1, 2, 5, 15.

### Right Hand

| X - Detected | |
|---|---|
| Frequency (Hz) | Y - Harmonic |
| 61 | 0 |
| 76.3 | 1 |
| 45.8 | −0.99 = −1 |
| 91.6 | 2 |
| 137 | 4.96 = 5 |
| 290 | 14.96 = 15 |

2. Female subject, 6 years old, Nov. 21, 2017, 4 p.m., Lima, Perú. Five (5) normal harmonics were detected using the right hand: 0, 1, 2, 3, 4.

| X - Detected | |
|---|---|
| Frequency (Hz) | Y - Harmonic |
| 61.0 | 0 |
| 76.3 | 1 |
| 91.6 | 2 |
| 107 | 3 |
| 122 | 4 |

3. Male subject, 9 years old, Jan. 3, 2018, 12 p.m., Barcelona, Spain. Twelve (12) normal harmonics were detected using the right hand: −5, 8, 11, 12, 13, 14, 15, 16, 17, 18, 19, 20.

| X - Detected | |
|---|---|
| Frequency (Hz) | Y - Harmonic |
| 290 | 14.96 = 15 |
| 320 | 16.92 = 17 |
| 259 | 12.94 = 13 |
| 183 | 7.97 = 8 |
| 229 | 10.98 = 11 |
| 351 | 18.95 = 19 |
| 244 | 11.96 = 12 |
| 275 | 13.98 = 14 |
| 366 | 19.93 = 20 |
| 305 | 15.94 = 16 |
| 336 | 17.97 = 18 |
| 137 | 4.96 = 5 |

4. Male subject, 6 years old, Jan. 2, 2018, 3 p.m., Lima, Perú. Eleven (11) normal harmonics were detected using the right hand: −1, 1, 2, 3, 4, 5, 7, 8, 9, 10, 11. Twenty-two (22) normal harmonics were detected using the left hand: 0, 1, 3, 4, 5, 6, 7, 8, 9, 10, 13, 14, 15, 18, 23, 25, 27, 28, 29, 31, 33, 41.

### Left Hand

| X - Detected | |
|---|---|
| Frequency (Hz) | Y - Harmonic |
| 290 | 14.96 = 15 |
| 137 | 4.96 = 5 |
| 259 | 12.94 = 13 |
| 488 | 27.90 = 28 |
| 534 | 30.91 = 31 |
| 443 | 24.96 = 25 |
| 687 | 40.91 = 41 |
| 580 | 33.92 = 33 |
| 504 | 28.95 = 29 |
| 473 | 26.92 = 27 |
| 275 | 13.98 = 14 |
| 214 | 10 |
| 198 | 8.95 = 9 |
| 183 | 7.97 = 8 |
| 153 | 6.01 = 6 |
| 336 | 17.97 = 18 |
| 412 | 22.94 = 23 |
| 122 | 3.98 = 4 |
| 168 | 6.99 = 7 |
| 61 | 0 |
| 76.3 | 1 |
| 107 | 3 |

### Right Hand

| X - Detected | |
|---|---|
| Frequency (Hz) | Y - Harmonic |
| 229 | 10.98 = 11 |
| 107 | 3 |
| 122 | 3.98 = 4 |
| 91.6 | 2 |
| 168 | 6.99 = 7 |
| 76.3 | 1 |
| 137 | 4.96 = 5 |
| 214 | 10 |
| 198 | 8.95 = 9 |
| 183 | 7.97 = 8 |
| 45.8 | −0.99 = −1 |

In the case of healthy female children, for the female subject of 3 years old in Case 1 only 6 harmonics appeared (−1, 0, 1, 2, 5, 15) and in Case 2 (6 years old) 5 harmonics appeared (0, 1, 2, 3, 4), with various frequencies in common and no more than 6 low-frequency harmonics (test with the right hand). In Case 3, the 9 years old child showed 12 normal harmonics which is a greater number of harmonics than the number obtained for younger children as explained above. In Case 4, the 6 years old girl (left-handed) showed 22 normal harmonic frequencies versus 11 normal harmonic frequencies for the right hand.

Adults and Children with Health Conditions:

1. Female subject, 80 years old, Nov. 16, 2017, 9 p.m., Lima, Perú. Diagnostic of consciousness disorder and weakness in hemi-right body (hemiplegia). Emergency Hospital “Casimiro Ulloa”, Dr. Edmundo López, Lima Perú. Fifteen (15) normal harmonics were detected: 2, 7, 9, 11, 17, 19, 20, 21, 22, 24, 25, 26, 29, 31, 33. Three (3) abnormal frequencies were detected: 56.86, 50.84, 35.88.

| X - Detected | |
|---|---|
| Frequency (Hz) | Y - Harmonic |
| 168 | 6.99 = 7 |
| 198 | 8.95 = 9 |
| 91.6 | 2 |
| 229 | 10.98 = 11 |
| 351 | 18.95 = 19 |
| 320 | 16.92 = 17 |
| 381 | 20.91 = 21 |
| 397 | 21.96 = 22 |
| 366 | 19.93 = 20 |
| 565 | 32.94 = 33 |
| 443 | 24.96 = 25 |
| 931 | 56.86 |
| 534 | 30.91 = 31 |
| 839 | 50.84 |
| 504 | 28.95 = 29 |
| 458 | 25.94 = 26 |
| 610 | 35.88 |
| 427 | 23.92 = 24 |

2. Female subject, 27 years old, Dec. 8, 2017, 10:30 a.m., Lima, Perú. Diagnostic of epilepsy since 11 years old. Twenty seven (27) normal harmonics were detected: 5, 7, 9, 10, 11, 14, 15, 17, 19, 20, 21, 22, 23, 24, 25, 26, 27, 28, 29, 31, 33, 35, 36, 37, 41, 42, 45. Eleven (11) abnormal frequencies were detected: 42.61, 48.86, 42.87, 39.86, 34.4, 51.83, 49.86, 47.84, 47.12, 50.84, 60.84. Note: repeated frequencies were not considered. On the first test, the subject had previously experienced spams, so that in the second test after one (1) hour an additional abnormal frequency was obtained.

| X - Detected | |
|---|---|
| Frequency (Hz) | |
| First Test | Y - Harmonic |
| 588 | 34.4 |
| 504 | 29 |
| 168 | 7 |
| 214 | 10 |
| 229 | 11 |
| 137 | 5 |
| 473 | 27 |
| 381 | 21 |
| 565 | 33 |
| 320 | 17 |
| 488 | 28 |
| 351 | 19 |
| 397 | 22 |
| 595 | 35 |
| 671 | 39.86 |
| 717 | 42.87 |
| 198 | 9 |
| 412 | 23 |
| 534 | 31 |
| 687 | 41 |
| 275 | 14 |
| 443 | 25 |
| 290 | 15 |
| 778 | 46.86 |
| 748 | 45 |
| 702 | 42 |
| 610 | 36 |
| 366 | 20 |
| 713 | 42.61 |
| 626 | 37 |

| X - Detected | |
|---|---|
| Frequency (Hz) | |
| Second Test | Y - Harmonic |
| 854 | 51.83 |
| 824 | 49.86 |
| 793 | 47.84 |
| 782 | 47.12 |
| 519 | 30 |
| 839 | 50.84 |
| 992 | 60.84 |

3. Male Subject, 58 years old, Jan. 3, 2018, 11 a.m. Diagnostic of Parkinson, “Hipolito Unanue” Hospital, Dr. Carlos Florez, Lima Perú. Eleven (11) normal harmonics were detected: 2, 3, 4, 5, 6, 7, 8, 9, 10, 11, 13.

| X - Detected | |
|---|---|
| Frequency (Hz) | Y - Harmonic |
| 198 | 8.95 = 09 |
| 153 | 6.01 = 06 |
| 137 | 4.96 = 05 |
| 91.6 | 02 |
| 122 | 3.98 = 04 |
| 168 | 6.99 = 7 |
| 214 | 10 |
| 183 | 7.97 = 8 |
| 229 | 10.98 = 11 |
| 259 | 12.94 = 13 |
| 107 | 3 |

4. Female Subject, 27 years old, Jan. 3, 2018, 11:30 a.m. Diagnostic of epilepsy, “Hipolito Unanue” Hospital, Dr. Carlos Florez, Lima Perú. Fourteen (14) normal harmonics were detected: 5, 6, 7, 8, 9, 10, 11, 12, 13, 14, 15, 16, 18, 19.

| X - Detected | |
|---|---|
| Frequency (Hz) | Y - Harmonic |
| 183 | 7.97 = 8 |
| 275 | 13.98 = 14 |
| 259 | 12.94 = 13 |
| 351 | 18.95 = 19 |
| 229 | 10.98 = 11 |
| 214 | 10 |
| 198 | 8.95 = 9 |
| 153 | 6.01 = 6 |
| 290 | 14.96 = 15 |
| 244 | 11.96 = 12 |
| 168 | 6.99 = 7 |
| 137 | 4.96 = 5 |
| 305 | 15.94 = 16 |
| 336 | 17.97 = 18 |

5. Female Subject, 72 years old, Jan. 4, 2018, 11:30 a.m. Diagnostic of hypertension, Lima, Perú. Sixteen (16) normal harmonics were detected: 8, 9, 10, 13, 16, 18, 19, 21, 23, 24, 27, 28, 30, 33, 35, 41. Six (6) abnormal frequencies were detected: 51.83, 46.86, 42.87, 38.88, 31.89, 45.88.

| X - Detected | |
|---|---|
| Frequency (Hz) | Y - Harmonic |
| 198 | 8.95 = 9 |
| 214 | 10 |
| 412 | 22.94 = 23 |
| 381 | 20.91 = 21 |
| 351 | 18.95 = 19 |
| 259 | 12.94 = 13 |
| 305 | 15.94 = 16 |
| 183 | 7.97 = 8 |
| 854 | 51.83 |
| 778 | 46.86 |
| 473 | 26.92 = 27 |
| 427 | 23.92 = 24 |
| 717 | 42.87 |
| 656 | 38.88 |
| 549 | 31.89 |
| 687 | 40.91 = 41 |
| 565 | 32.94 = 33 |
| 336 | 17.97 = 18 |
| 488 | 27.90 = 28 |
| 763 | 45.88 |
| 519 | 29.93 = 30 |
| 595 | 34.90 = 35 |

6. Female Subject, 43 years old, Jan. 9, 2018, 11:30 a.m. Diagnostic of depression. “Hipolito Unanue” Hospital, Dr. Eduardo Rodriguez (Psychiatrist), Lima, Perú. Twelve (12) normal harmonics were detected: 7, 9, 10, 11, 12, 13, 14, 15, 16, 18, 19. One (1) abnormal frequencies was detected: 21.17.

| X - Detected | |
|---|---|
| Frequency (Hz) | Y - Harmonic |
| 275 | 13.98 = 14 |
| 214 | 10 |
| 244 | 11.96 = 12 |
| 259 | 12.94 = 13 |
| 168 | 6.99 = 7 |
| 320 | 16.92 = 17 |
| 229 | 10.98 = 11 |
| 305 | 15.94 = 16 |
| 198 | 8.95 = 9 |
| 336 | 17.97 = 18 |
| 351 | 18.95 = 19 |
| 290 | 14.96 = 15 |
| 385 | 21.17 |

7. Female Subject, 55 years old, Jan. 12, 2018, 12 a.m. Diagnostic of minor cognitive deterioration. “Hipolito Unanue” Hospital, Dr. Carlos Florez, Lima, Perú. Fourteen (14) normal harmonics were detected using the right hand: 0, 1, 2, 3, 4, 5, 6, 7, 8, 11, 12, 13, 14, 15. Twenty-two (22) normal harmonics were detected using the left hand: −1, 0, 1, 2, 3, 4, 5, 6, 7, 8, 10, 11, 12, 13, 14, 15, 16, 17, 18, 20, 23, 25. One (1) abnormal frequency was detected using the left hand: 8.43.

### Right Hand

| X - Detected | |
|---|---|
| Frequency (Hz) | Y - Harmonic |
| 91.6 | 2 |
| 76.3 | 1 |
| 61.8 | 0 |
| 137 | 4.96 = 5 |
| 107 | 3 |
| 122 | 3.98 = 4 |
| 229 | 10.98 = 11 |
| 153 | 6.01 = 6 |
| 168 | 6.99 = 7 |
| 259 | 12.94 = 13 |
| 275 | 13.98 = 14 |
| 183 | 7.97 = 8 |
| 290 | 14.96 = 15 |
| 244 | 11.96 = 12 |

### Left Hand

| X - Detected | |
|---|---|
| Frequency (Hz) | Y - Harmonic |
| 91.6 | 2 |
| 153 | 6.01 = 6 |
| 107 | 3 |
| 61.0 | 0 |
| 190 | 8.43 |
| 45.8 | −0.99 = −1 |
| 76.3 | 1 |
| 16.8 | 6.99 = 7 |
| 13.7 | 4.96 = 5 |
| 229 | 10.98 = 11 |
| 122 | 3.98 = 4 |
| 259 | 12.94 = 13 |
| 320 | 16.92 = 17 |
| 244 | 11.96 = 12 |
| 305 | 15.94 = 16 |
| 275 | 13.98 = 14 |
| 290 | 14.96 = 15 |
| 214 | 10 |
| 183 | 7.97 = 8 |
| 366 | 19.93 = 20 |
| 443 | 24.96 = 25 |
| 412 | 22.94 = 23 |
| 336 | 17.97 = 18 |

8. Male Subject, 81 years old, Jan. 31, 2018, 12 a.m. Diagnostic of Parkinson), Peruvian Air Force Hospital, Dr. Luis Tudela, Lima, Perú. Sixteen (16) normal harmonics were detected: 16, 20, 23, 25, 27, 29, 30, 31, 33, 34, 35, 37, 38, 41, 45, 50. Fifteen (15) abnormal frequency were detected: 50.84, 60.84, 39.86, 42.87, 41.89, 45.88, 46.86, 47.84, 56.86, 52.87, 53.85, 66.60, 58.82, 43.85, 35.88.

### Right Hand

| X - Detected | |
|---|---|
| Frequency (Hz) | Y - Harmonic |
| 504 | 28.95 = 29 |
| 839 | 50.84 |
| 626 | 36.92 = 37 |
| 687 | 40.91 = 41 |
| 519 | 29.93 = 30 |
| 641 | 37.90 = 38 |
| 443 | 24.96 = 25 |
| 671 | 39.86 |
| 717 | 42.87 |
| 992 | 60.84 |
| 534 | 30.91 = 31 |
| 824 | 49.86 = 50 |
| 809 | 48.88 |
| 778 | 46.86 |
| 702 | 41.89 |
| 793 | 47.84 |
| 931 | 56.86 |
| 565 | 32.94 = 33 |
| 870 | 52.87 |
| 885 | 53.85 |
| 595 | 34.90 = 35 |
| 748 | 44.90 = 45 |
| 534 | 30.91 = 31 |
| 473 | 26.92 = 27 |
| 900 | 54.83 |
| 1080 | 66.60 |
| 961 | 58.82 |
| 366 | 19.93 = 20 |
| 732 | 43.85 |
| 305 | 15.94 = 16 |
| 763 | 45.88 |
| 610 | 35.88 |
| 580 | 33.92 = 34 |
| 412 | 22.94 = 23 |

9. Female Subject, 87 years old, Feb. 2, 2018, 3:00 p.m. Diagnostic: Beginning of Parkinson and Alzheimer, Lima, Perú. Six (6) normal harmonics were detected using the right hand: −1, 1, 2, 3, 4, 5. Nine (9) normal harmonics were detected using the left hand: −1, 0, 1, 2, 3, 4, 5, 6, 8.

### Right Hand

| X - Detected | |
|---|---|
| Frequency (Hz) | Y - Harmonic |
| 76.3 | 1 |
| 91.6 | 2 |
| 45.8 | −0.99 = −1 |
| 107 | 3 |
| 137 | 4.96 = 5 |
| 122 | 3.98 = 4 |

### Left Hand

| X - Detected | |
|---|---|
| Frequency (Hz) | Y - Harmonic |
| 76.3 | 1 |
| 61 | 0 |
| 107 | 3 |
| 122 | 3.98 = 4 |
| 137 | 4.96 = 5 |
| 45.8 | −0.99 = −1 |
| 91.6 | 2 |
| 153 | 6.01 = 6 |
| 183 | 7.97 = 8 |

10. Female Subject, 63 years old, Feb. 9, 2018, 5:30 p.m. Diagnostic of Migraine/Glaucoma, Trujillo, Perú. Eight (8) normal harmonics were detected using the left hand: 1, 3, 4, 5, 6, 7, 8, 9. Eleven (11) normal harmonics were detected using the right hand: −1, 0, 1, 3, 5, 7, 8, 9, 11, 121, 138. Four (4) abnormal frequencies were detected using the right hand: 185.55, 138.49, 136.53, 204.57.

### Left Hand

| X - Detected | |
|---|---|
| Frequency (Hz) | Y - Harmonic |
| 122 | 3.98 = 4 |
| 137 | 4.96 = 5 |
| 168 | 6.99 = 7 |
| 107 | 3 |
| 76.3 | 1 |
| 198 | 8.95 = 9 |
| 153 | 6.01 = 6 |
| 183 | 7.97 = 8 |

### Right Hand

| X - Detected | |
|---|---|
| Frequency (Hz) | Y - Harmonic |
| 198 | 8.95 = 9 |
| 168 | 6.99 = 7 |
| 183 | 7.97 = 8 |
| 2.90K | 185.55 |
| 1.85K | 120.91 = 121 |
| 2.11K | 137.90 = 138 |
| 2.18K | 138.49 |
| 2.15K | 136.53 |
| 3.13K | 204.57 |
| 229 | 10.98 = 11 |
| 76.3 | 1 |
| 45.8 | −0.99 = −1 |
| 61.0 | 0 |
| 107 | 3 |
| 137 | 4.96 = 5 |

11. Male Subject, 86 years old, Feb. 10, 2018, 5:00 p.m. Diagnostic of Alzheimer, Trujillo city, Perú. Six (6) normal harmonics were detected using the right hand: −1, 1, 2, 3, 4, 5. Seven (7) normal harmonics were detected using the left hand: −1, 0, 1, 2, 3, 5, 7.

### Right Hand

| X - Detected | |
|---|---|
| Frequency (Hz) | Y - Harmonic |
| 76.3 | 1 |
| 45.8 | −0.99 = −1 |
| 107 | 3 |
| 91.6 | 2 |
| 122 | 3.98 = 4 |
| 137 | 4.96 = 5 |

### Left Hand

| X - Detected | |
|---|---|
| Frequency (Hz) | Y - Harmonic |
| 76.3 | 1 |
| 45.8 | −0.99 = −1 |
| 107 | 3 |
| 91.6 | 2 |
| 61 | 0 |
| 137 | 4.96 = 5 |
| 168 | 6.99 = 7 |

12. Female Subject, 77 years old, Mar. 2, 2018, 12 p.m. Diagnostic of Alzheimer, Lima, Perú. Five (5) normal harmonics were detected using the right hand: −1, 0, 1, 3, 5. Six (6) normal harmonics were detected using the left hand: −1, 0, 1, 2, 3, 5.

### Right Hand

| X - Detected | |
|---|---|
| Frequency (Hz) | Y - Harmonic |
| 76.3 | 1 |
| 45.8 | −0.99 = −1 |
| 107 | 3 |
| 61 | 0 |
| 137 | 4.96 = 5 |

### Left Hand

| X - Detected | |
|---|---|
| Frequency (Hz) | Y - Harmonic |
| 76.3 | 1 |
| 45.8 | −0.99 = −1 |
| 107 | 3 |
| 91.6 | 2 |
| 61 | 0 |
| 137 | 4.96 = 5 |

13. Female Subject, 59 years old, Mar. 6, 2018, 11:30 a.m. Diagnostic of Alzheimer, Dr. Tudela, Peruvian Air Force Hospital, Lima, Perú. Two (2) normal harmonics were detected using the left hand: 38, 64. Eleven (11) abnormal frequencies were detected using the left hand: 39.86, 47.84, 49.86, 51.83, 48.88, 69.86, 53.85, 47.84, 59.86, 57.84, 75.81. Eight (8) normal harmonics were detected using the right hand: 6, 8, 10, 12, 14, 22, 24, 34. Two (2) abnormal frequencies were detected using the right hand: 39.86, 41.89.

### Left Hand

| X - Detected | |
|---|---|
| Frequency (Hz) | Y - Harmonic |
| 671 | 39.86 |
| 793 | 47.84 |
| 824 | 49.86 |
| 641 | 37.90 = 38 |
| 854 | 51.83 |
| 809 | 48.88 |
| 1.04 KZ | 63.98 = 64 |
| 1.13 KZ | 69.86 |
| 885 | 53.85 |
| 793 | 47.84 |
| 977 | 59.86 |
| 946 | 57.84 |
| 1.16 KZ | 75.81 |

### Right Hand

| X - Detected | |
|---|---|
| Frequency (Hz) | Y - Harmonic |
| 275 | 13.98 = 14 |
| 244 | 11.96 = 12 |
| 183 | 7.97 = 8 |
| 153 | 6.01 = 6 |
| 214 | 10 |
| 671 | 39.86 |
| 702 | 41.89 |
| 427 | 23.92 = 24 |
| 397 | 21.96 = 22 |
| 580 | 33.92 = 34 |

14. Male Subject, 21 years old, Mar. 14, 2018, 11:00 a.m. Diagnostic of Down Syndrome, Lima, Perú. Sixteen (16) normal harmonics were detected using the right hand: 10, 12, 14, 16, 17, 18, 19, 20, 22, 23, 24, 26, 28, 30, 33, 38. Sixteen (16) normal harmonics were detected using the left hand: 6, 7, 8, 10, 11, 12, 13, 14, 16, 17, 18, 19, 20, 22, 23, 30.

### Right Hand

| X - Detected | |
|---|---|
| Frequency (Hz) | Y - Harmonic |
| 305 | 15.94 = 16 |
| 427 | 23.92 = 24 |
| 336 | 17.97 = 18 |
| 397 | 21.96 = 22 |
| 519 | 29.93 = 30 |
| 458 | 25.94 = 26 |
| 488 | 27.90 = 28 |
| 366 | 19.93 = 20 |
| 275 | 13.98 = 14 |
| 580 | 33.92 = 34 |
| 351 | 18.95 = 19 |
| 244 | 11.96 = 12 |
| 320 | 16.92 = 17 |
| 412 | 22.94 = 23 |
| 214 | 10 |
| 641 | 37.98 = 38 |

### Left Hand

| X - Detected | |
|---|---|
| Frequency (Hz) | Y - Harmonic |
| 336 | 17.97 = 18 |
| 183 | 7.97 = 8 |
| 244 | 11.96 = 12 |
| 275 | 13.98 = 14 |
| 259 | 12.94 = 13 |
| 366 | 19.93 = 20 |
| 229 | 10.98 = 11 |
| 397 | 21.96 = 22 |
| 214 | 10 |
| 153 | 6.01 = 6 |
| 305 | 15.94 = 16 |
| 320 | 16.92 = 17 |
| 519 | 29.93 = 30 |
| 351 | 18.95 = 19 |
| 412 | 22.94 = 23 |
| 168 | 6.99 = 7 |

In Case 1, the 80 years old subject with a diagnosis of “consciousness disorder and hemi-right body weakness” showed the following harmonic multiples for 15.3 Hz: 2, 7, 9, 11, 17, 19, 20, 21, 22, 24, 25, 26, 29, 31, 33. These multiples are common and consistent to the experienced by subjects in healthy condition with the exception of three frequencies: 931 Hz, 839 Hz and 610 Hz that produce the abnormal harmonics of 56.86, 50.84 and 35.88 that cannot be approximated or rounded to the integers of 57, 51 and 36 respectively, since they are below the values for integer approximation and rounding. It should be noted that in some calculations of the harmonic's formula like for example the number 229 yields 10.98 which approaches 11. This is because in all cases the digit 9 positioned at the tenth of a decimal is considered as a requirement for approximating to the next upper integer. However, when this digit is smaller than 9 (like 8 as in the three frequencies above) the presence of some abnormality corresponding to the subject's illness or disease is indicated.

In case 2, the female subject suffers from epilepsy since age 11 and is now 27 years old currently under daily medication. The subject had an epileptic seizure in 2017 and now a days suffers from temporary spams. The normal harmonics detected in the experiments for this subject were: 5, 7, 9, 10, 11, 14, 15, 17, 19, 20, 21, 22, 23, 24, 25, 26, 27, 28, 29, 31, 33, 35, 36, 37, 41, 42, 45 while the 11 abnormal harmonics were detected: 42.61, 48.86, 42.87, 39.86, 34.4, 51.83, 49.86, 47.84, 47.12, 50.84, 60.84. Note that repeated frequencies were not considered. On the first experiment the subject previously suffered spams so that in the second test after 1 hour 6 additional abnormal frequencies were obtained.

In case 3, the male subject had been treated at the “Hipolito Unanue” Hospital during 5 years with medications for controlling the Parkinson disease having improved his health which was corroborated with the test performed where 11 normal harmonics were detected and none abnormal.

In case 4, the female subject suffers from epilepsy since birth and has been treated by the Department of Neurology at the “Hipolito Unanue” Hospital during the last couple of years and had an epileptic attack last year. Currently the subject shows a better health condition which was corroborated by the test obtaining 14 normal harmonics and none abnormal.

In case 5, the female subject was under daily medication (LOSACOR-D) for hypertension by medical prescription for over 5 years. In this case it was possible to detect 6 abnormal harmonic frequencies, an indication that this medication might be collaterally affecting the nervous system and a reassessment of the specialized treatment might be necessary.

In case 6, the female subject showed 11 normal harmonics and a single abnormal harmonic within the tolerable range for a person in a healthy condition. So, the problem is not due to an internal organic alteration, but rather due to some depressive psychological problem, according also to the medical specialist opinion.

In case 7, the female subject which had a diagnosis of mild cognitive impairment and was under daily medication to treat rheumatology obtained 14 normal harmonic frequencies with the right hand, 22 normal harmonicas with the left hand, and a single abnormal harmonic of 8.43, which is in the tolerable range.

In case 8, the male subject had a diagnosis of Parkinson with over years suffering from the disease. Seventeen (17) abnormal harmonic frequencies were detected and verified. These frequencies were fast and greater than 400 Hz while using the right hand.

In case 9, the female subject had an age of 87 years showing the beginning of Parkinson's and Alzheimer's, wherein reduced normal harmonic frequencies were detected corroborating a greater number of frequencies detected (09) using the left hand using the right (06). Likewise confirming as in case 6 (female subject with 97 years old) a lower presence of harmonic frequencies very much like children.

In case 10, eight (8) normal frequencies were detected for the female subject using the left hand during the test. However, 11 normal harmonics frequencies and 4 abnormal high frequencies were detected when using the right hand, which were detected consecutively in a burst. This corroborated the migraine pain suffered by the subject in the left lobule of the head, where she manifested chronic pain. Also, this case, unlike other cases, showed more frequencies than those registered in the left hand.

In case 11, the male subject only showed 6 and 7 normal harmonic frequencies which were detected using the right and the left hand, respectively, where the test was taken in the city of Trujillo. Alzheimer's disease appeared in 2012, in addition to Diabetes.

For Alzheimer's, the subject was under medication with Memantine Hydrochloride (Clauss) every 12 hrs. No abnormal frequencies were detected, while the subject showed distortion of reality and lack of memory.

In case 12, the female subject has been suffering from Alzheimer's disease for the last 8 years and she was tested using the left hand and the right hand with only 6 and 5 normal harmonic frequencies detected, respectively. The subject was medicated with levothyroxine, aspirin and quetiapine, showing muscular rigidity, loss of speech and memory. No abnormal frequencies were detected.

In case 13, the female subject had Alzheimer's disease and the test were performed on the left hand where 2 normal harmonic frequencies and 11 abnormal frequencies were detected, while 8 normal harmonic frequencies and 2 abnormal frequencies were also detected when using the right hand. The subject was under medication with rivastigmine and memantine. However, the medication used by the subject in the previous case was different and normal frequencies recorded, which could be analyzed and evaluated by the specialist to optimize the treatment and control of the disease.

In case 14, the male subject has down syndrome and did not register any abnormal frequency in the tests performed on both hands, indicating a good brain function since birth, unlike the other cases exposed with hemiplegia, epilepsy, Parkinson, Alzheimer and migraine, diseases that were developed by the subjects after birth.

The advantages of using this procedure to detect the normal harmonics of the first harmonic of the Earth's atmospheric electromagnetic resonance frequency (15.3 Hz) and the abnormal frequencies associated to Neurological diseases will serve neurologists as a guide for a better treatment to patients. Disappearance of the indicated frequencies corresponding to neurological diseases will indicate that the physician is providing a good treatment to the patients. Increase in the amount of these frequencies will indicate that the physician's treatment might not be the most appropriate.

### DISCUSSION

As previously explained, Earth's atmospheric electromagnetic resonance frequency (Schumann) is a biological constant that directly affects the hypothalamus of individuals and all mammals and thus, the health. The inventor discovered and detected that the first harmonic (15.3 Hz) of this frequency has the strongest intensity of all harmonics, having been verified this value remains constant in Lima, Barcelona and Jerusalem, thus inferring that it remains constant throughout the planet. Therefore, the method of the present invention is valid for its detection anywhere around the world.

The procedure according to the present invention was carried out voluntarily in healthy people, particular patients and patients from the “Casimiro Ulloa”, “Hipolito Unanue” and Fuerza Aerea del Perú Emergency Hospitals, in the presence of a neurologist and patient relatives. The results obtained with this procedure will be explained below.

People in good health condition show normal harmonic frequencies that are submultiples or multiples of the Earth's first harmonic resonance frequency of 15.3 Hz. These people can have as a tolerance one (1) abnormal frequency (understanding as abnormal frequency those frequencies that are not multiples of 15.3 Hz).

Depending on each person's body resistance, the number of normal harmonic frequencies submultiple or multiples of 15.3 Hz and abnormal frequencies, may vary in function of the age, the tests being performed with either the right or the left hand and the time of the day.

When the tests are performed in children and elder people in good health using the right hand, the amount of normal harmonic frequencies multiples of 15.3 Hz, do not exceed 6 where the numerical results are mostly exact (multiply whole numbers of 15.3 Hz).

A left-handed or right-handed person, when performing the test using the right hand will have an equal or less amount of normal harmonic frequencies that are multiples of 15.3 Hz, than when using the left hand, except for the case of chronic migraine.

In the invention, formula Y=(X−61)/15.3 is used, where X is the frequency detected and Y is the harmonic {dimensionless} multiple of 15.3 Hz that will not vary across the world. The value of 61 (Hz) is the detection constant of the Earth's first atmospheric electromagnetic frequency of resonance harmonic, of the (15.3 hz).

The tests performed were carried out each time for periods of approximately 2 minutes, in places free of possible interferences that can originate from industrial machines, electric devices and/or instruments and biomedical equipment and the like.

The tests performed indicate that even when a person in apparent good health could be taking medications to control the disease, if the medication is not adequate abnormal frequencies could be generated indicating that the treatment might not be the most appropriate. However, it has also been appreciated that in patients with chronic Parkinson under daily medication, the disease is controlled, and no abnormal frequencies recorded corroborating the good treatment provided by the specialist.

A person in good health condition or with a disease that is controlled with medication, will never generate abnormal frequencies and will only generate normal harmonic frequencies that are submultiple or multiples of 15.3 Hz, Earth's atmospheric electromagnetic resonant frequency first harmonic.

Psychological depression does not alter the normal frequency harmonics multiples of 15.3 Hz on a person that are indicators of good neurological health. People with some neurological disease have normal harmonic frequencies multiples of 15.3 Hz and abnormal frequencies that fall between the above-mentioned harmonic frequencies.

There is a correlation of the normal harmonic frequencies of 15.3 Hz detected in healthy people by age. Few frequencies are detected in children and elder people, whereas a greater amount is detected in adolescents and adult people. Thus, it can be inferred that the amount of detected frequencies is in direct function with the state of development and deterioration of cerebral function.

Children and adults with good health that perform increased intellectual and cognitive exercises may generate a greater amount of normal frequencies harmonics of 15.3 Hz with faster detection speeds.

The number of the normal harmonic frequencies of 15.3 Hz can be correlative from −1, 0, 1, 2, . . . n, and can also not be where some of these numbers are missing regardless of their position and cyclic repetition. Harmonic −1 represents the reference frequency 0 Hz (45.8 Hz as registered in the mini-oscilloscope). Harmonic o indicates the Earth's electromagnetic atmospheric resonant frequency of 7.83 Hz (61 Hz as registered in the mini-oscilloscope). Harmonic 1 represents the 15.3 Hz, first harmonic of Earth's electromagnetic atmospheric resonant frequency of 7.83 Hz (76.3 Hz as registered in the mini-oscilloscope).

The tests must be performed on both the left hand and the right hand, each one time in order to cover all the possibilities of detecting any neurological disease.

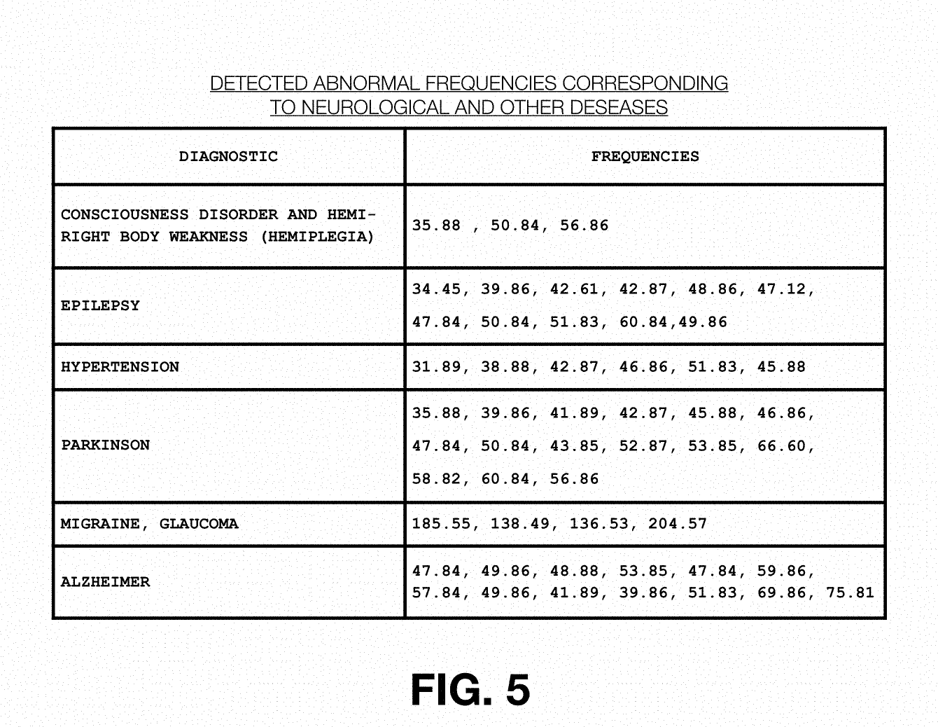

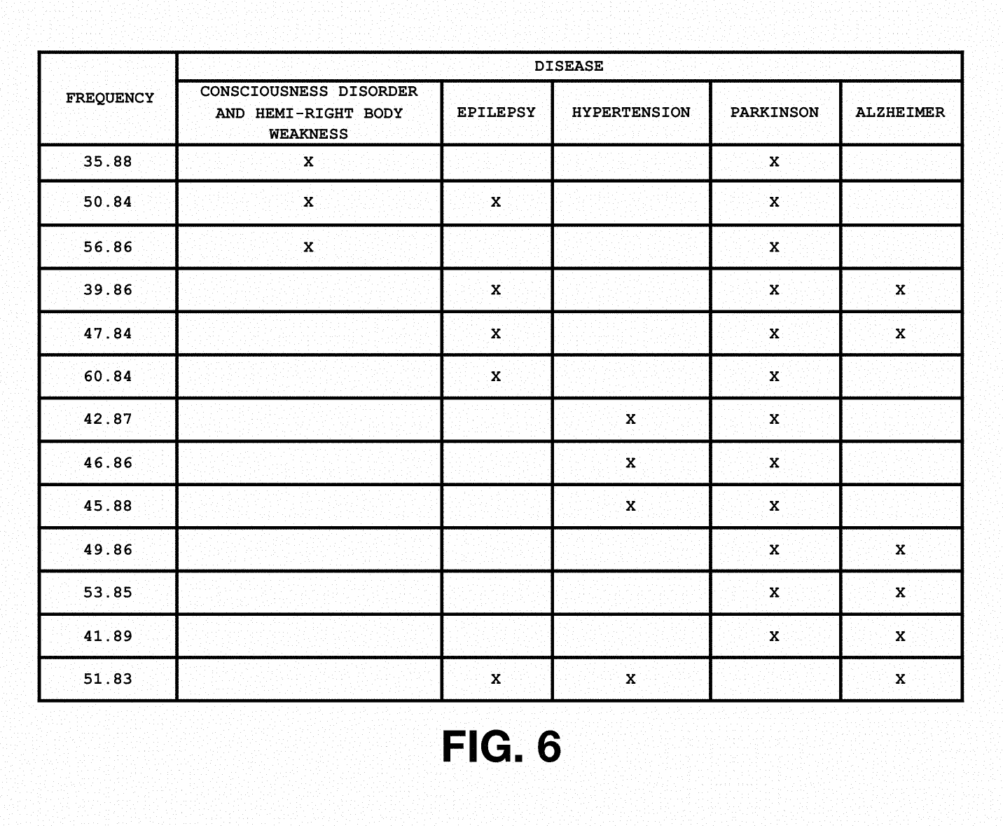

Neurological and other type of diseases like hypertension, have common frequencies as shown in FIG. 6, indicating that medical treatments and medication could be effective between them. Moreover, it was determined that several diseases can be correlated to certain frequencies as shown in FIG. 5.

In patients with Alzheimer, the presence of abnormal frequencies has been registered if the medication is not adequate. In contrast, other patients with different medication have not registered said frequencies.

In the case of subjects with down syndrome, no abnormal frequencies were detected, and the brain corresponded to the brain of a healthy person since birth but with reduced learning ability. However, abnormal frequencies were detected in healthy people who developed neurological diseases throughout their lives.

The detection steps of these normal and abnormal harmonic frequencies according to the present invention, can be automated with the assistance of a frequency recording equipment that integrates real-time registration, storage and print the data of each patient. This instrument combined with the use of another frequency generating equipment, which applies the principle of North American Dr. Hulda Clark related to the electrical equilibrium of the pH, could counter, decrease and/or control neurological abnormal frequencies in such a way to “tune” the cerebral pulses that pass through the hypothalamus.

Additional statistics results for the experiments performed according to the present invention were not necessary, since the main objective of the procedure was the detection and identification of the normal harmonics of Earth's electromagnetic atmospheric resonant frequency first harmonic of 15.3 Hz and abnormal frequencies corresponding to neurological diseases.

Although the invention has been described in conjunction with specific embodiments, it is evident that many alternatives and variations will be apparent to those skilled in the art in light of the foregoing description. Accordingly, the invention is intended to embrace all of the alternatives and variations that fall within the spirit and scope of the appended claims.

1-7. (canceled)

8. A method for detecting on an individual, normal harmonics from the first harmonic of the Earth's atmospheric electromagnetic resonant frequency and abnormal frequencies corresponding to neurological diseases, the method comprising:
connecting a negative port of an oscilloscope to ground;
contacting a positive port of the oscilloscope to a hand of the individual;
turning on said oscilloscope;
reading frequencies shown on said oscilloscope for a predetermined amount of time;
applying to each of the frequencies read the following equation:
$$Y = \frac{( X - 61 )}{15.3} ,$$
where X is the frequency read in Hz and Y is the harmonic corresponding to the detected frequency; and
detecting on said individual, normal harmonics and abnormal frequencies corresponding to neurological diseases, wherein:
a normal harmonic is detected if the result Y is an integer or a value z.9w, where z and w is a number greater than or equal to zero and w is a number between 0-9, and
an abnormal frequency is detected if the result Y is a value z.sw, where z is a number greater than or equal to zero, s is number between 0-8 and w is a number between 0-9.

9. The method of claim 8, wherein said predetermined amount of time is 2 minutes.

10. The method of claim 8, wherein the positive port of the oscilloscope is contacted with a left hand of the individual.

11. The method of claim 8, wherein the positive port of the oscilloscope is contacted with a right hand of the individual.

12. The method of claim 8, wherein the positive port of the oscilloscope is contacted with the hand of the individual via an electrical cable.

13. The method of claim 8, wherein the individual is seated, standing up or lying down during the frequency readings.

14. The method of claim 12, wherein said electrical cable has a sectional area between 0.3-3 mm and a length between 10-20 cm.

15. The method of claim 8, wherein a USB interface of said oscilloscope is connected to one of: a USB port of a computer and a power adapter.

16. The method of claim 15, wherein said power adapter down converts an input voltage to 5V_{dc}.

17. The method of claim 15, wherein said input voltage is provided by an electrical power network.

18. The method of claim 8, wherein said positive port of the oscilloscope contacts the hand of the individual by connecting an end of a first electrical cable to said positive port and the other end of said first electrical cable to a protoboard; and connecting an end of a second electrical cable to said protoboard in parallel to the other end of said first electrical cable and having the individual contact the other end of said second electrical cable.

19. The method of claim 18, wherein the negative port of said oscilloscope is connected to ground on said protoboard.

20. The method of claim 8, wherein the detection is performed on a place free of electromagnetic interference.

21. The method of claim 8, wherein said oscilloscope has a maximum input voltage of 80 Vₚₚ, a maximum sampling rate of 1 Msps/12 bit, an input resistance greater than 500 kohms, a transverse sensitivity of 1 μS/Div-10S/Div, a vertical sensitivity of 10 mv/Div-10 v/Div (x1 probe) and 0.5 v/Div-10 v/Div (x10 probe).
